(19) United States
(12) Patent Application Publication
(10) Pub. No.: US 2022/0064453 A1
(43) Pub. Date: Mar. 3, 2022

(54) COMPOSITION FOR DYEING BENTONITE AND METHOD FOR DYEING BENTONITE

(71) Applicant: Korea Institute of Geoscience And Mineral Resources, Daejeon (KR)

(72) Inventors: Jae Hwan KIM, Pohang-si (KR); Chang Yun PARK, Seoul (KR); Sung Man SEO, Pohang-si (KR); Il Mo KANG, Seoul (KR); Ki Min ROH, Daejeon (KR)

(73) Assignee: Korea Institute Of Geoscience And Mineral Resources, Daejeon (KR)

(21) Appl. No.: 17/362,057

(22) Filed: Jun. 29, 2021

(30) Foreign Application Priority Data
Sep. 2, 2020 (KR) 10-2020-0111483

(51) Int. Cl. C09B 65/00

(52) U.S. Cl. CPC C09B 65/00

(57) ABSTRACT

A composition for dyeing a bentonite, which includes a plant-derived polyphenol, and a method for dyeing a bentonite using the composition are provided. A composition for dyeing a bentonite, which includes a polyphenol such as lignin, pectin, tannin, and catechin, as a plant-derived material, and a method for dyeing a bentonite using the composition are provided.

### CROSS-REFERENCE TO RELATED APPLICATION

This application claims the benefit of Korean Patent Application No. 10-2020-0111483, filed on Sep. 2, 2020, in the Korean Intellectual Property Office, the disclosure of which is incorporated herein by reference.

### BACKGROUND

### 1. Field of the Invention

The present disclosure relates to a composition for dyeing a clay, which includes a plant-derived material, and a method for dyeing a clay using the plant-derived material. More specifically, the present disclosure relates to a composition for dyeing a clay, which includes a polyphenol such as lignin, pectin, tannin, and catechin as the plant-derived material, and a mordant, and a method for dyeing a bentonite using the composition.

### 2. Description of the Related Art

Dyeing is divided into artificial dyeing using synthetic dyes and natural dyeing using natural dyes. The synthetic dyes have been mostly used to obtain various colors and excellent dyeing results, but it is found that the synthetic dyes release various environmental pollutants in use, and textile products dyed by the synthetic dyes has adverse effects on the human body, such as atopic dermatitis. On the other hand, unlike artificial dyeing using the synthetic dyes, natural dyeing, which mainly uses barks, stems, fruits, or roots of plants, has advantages such as harmless effects on the human body, readily available raw materials, easy dye extraction, and a simple dyeing process.

Accordingly, natural dyeing using substances derived from natural products such as plants is actively used in industry by increasing its dyeability. There are problems such as low durability or change in properties of dyed substances by processing or manipulation to increase dyeability of substances to be naturally dyed. In order to solve these problems, technology development for standardization, automation, and mass production of natural dyeing technology is required.

A bentonite is a type of clay mineral containing montmorillonite as its main constituent mineral, and is known to be widely buried in the East Coast of Korea. The bentonite is an aggregate of fine colloidal substances so that when put in water, the bentonite is absorbed and swollen, shows high viscosity, and has a high cation exchange ability compared to other clay minerals, so its uses are very diverse. Thus, the bentonite is mainly used as a binder for castings, varnish for drilling, and for civil engineering and construction. By including the bentonite in cosmetics or beauty packs, the bentonite is used for skin waste removal, skin regeneration, or skin diseases such as melasma, freckles, acne, blemishes, dead skin cells, and atopy. In addition, a natural bentonite is one of the colorless clays, and may be colored and used as a cosmetic raw material.

A product for dyeing animal or human hair using a metal salt as an active mordant, and a tea pigment which is a tea polyphenol or an oxidation product thereof as an active dye that exists in nature, is provided in Korean Registration Patent No. 10-1621032, while the present disclosure provides a composition for dyeing a clay such as a bentonite using natural polyphenols and mordants.

### SUMMARY

It is found that a dyeing composition including a plant-derived polyphenol and a mordant dyes a bentonite that is one of clay minerals, and is used as a raw material for increasing a coloring effect, so that the present disclosure is completed.

According to an aspect of the present disclosure, there is provided a composition for dyeing a bentonite, the composition including a polyphenol and a mordant.

According to an embodiment of the present disclosure, the polyphenol may be at least one of lignin, pectin, tannin, and catechin.

According to an embodiment of the present disclosure, the mordant may be at least one of Fe³⁺, Fe²⁺ and Al³⁺.

According to an embodiment of the present disclosure, a molar ratio of the polyphenol to the mordant may range from 1:1 to 1:10.

According to another aspect of the present disclosure, there is provided a bentonite dyed by a composition for dyeing a bentonite, the composition including a polyphenol and a mordant.

According to an embodiment of the present disclosure, the dyed bentonite may have a crystalline spherical shape and a diameter of 20 to 50 micrometers (μm).

According to another aspect of the present disclosure, there is provided a cosmetic composition containing a bentonite dyed by a composition for dyeing a bentonite, the composition including a polyphenol and a mordant.

According to an embodiment of the present disclosure, the dyed bentonite may have a crystalline spherical shape and a diameter of 20 to 50 μm.

According to another aspect of the present disclosure, there is provided a method for dyeing a bentonite, the method including steps of separating and purifying a bentonite; mixing a polyphenol and a mordant to prepare a solution; and stirring a mixed solution of the polyphenol and the mordant and a separated and purified bentonite.

According to an embodiment of the present disclosure, the separated and purified bentonite may have a particle size of 0.01 to 100 μm.

According to an embodiment of the present disclosure, the bentonite may be included in an amount of 1 to 20% by weight (wt %) based on 100 wt % of the mixed solution of the polyphenol and the mordant.

However, aspects of the present disclosure are not limited to the one set forth herein, and other aspects not mentioned herein would be clearly understood by one of ordinary skill in the art from the following description.

According to example embodiments, a composition for dyeing a bentonite of the present disclosure may be used to variously change the color of a natural bentonite to yellow, blue, or brown, and mineralogical composition properties of the bentonite may be maintained while enhancing a coloring effect.

According to example embodiments, in particular, since a bentonite dyed by the composition for dyeing a bentonite of the present disclosure has a crystalline spherical shape and a uniform diameter upon spray drying, skin texture is good, and therefore the dyed bentonite is suitable as a cosmetic raw material.

The effects of the present disclosure are not limited to the above effects, and should be understood to include all effects that can be inferred from the configuration of the invention described in the detailed description or claims of the present disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1A shows a white natural bentonite after purification, and FIG. 1B shows a bentonite dyed with a pine needle extract and Fe³⁺.

FIG. 2 shows various colors of a bentonite dyed with a solution prepared by mixing a polyphenol having a molarity of 10 mM and a mordant having a molarity of 100 mM.

FIG. 3 shows various colors of a bentonite dyed with a solution prepared by mixing a polyphenol having a weight of 10 mg and a mordant having a molarity of 50 mM.

FIG. 4 illustrates a result of XRD analysis in order to measure a change in a mineralogical composition of a bentonite colored by the composition for dyeing a bentonite containing a polyphenol and a mordant of the present disclosure.

FIG. 5 shows a CIE L*a*b* color space graph.

### DETAILED DESCRIPTION

Hereinafter, example embodiments will be described in detail with reference to the accompanying drawings. However, as various changes may be applied to the example embodiments, the right scope of patent application is not restricted or limited by the example embodiments. It should be understood that all modifications, equivalences, or substitutions for the example embodiments are included in the right scope.

Terms used in the example embodiments are used for the purposes of illustration only, but should not be interpreted as intended to limit the right scope. An expression used in the singular encompasses the expression in the plural, unless it has a clearly different meaning in the context. In this specification, it should be understood that a term such as “including” or “having” is used to specify the presence of features, numbers, steps, operations, constituent elements, parts, or any combination thereof described in the specification, but it does not preclude the possibility of the presence or addition of one or more other features, numbers, steps, operations, constituent elements, parts, or any combination thereof.

Unless otherwise defined, all terms (including technical and scientific terms) used herein have the same meaning as commonly understood by one of ordinary skill in the art to which the example embodiments belong. It will be further understood that terms, such as those defined in commonly used dictionaries, should be interpreted as having a meaning that is consistent with their meaning in the context of the relevant art and will not be interpreted in an idealized or overly formal sense unless clearly defined in the present application.

In describing the example embodiments with reference to the accompanying drawings, like elements will be referenced by like reference numerals or signs regardless of the drawing numbers, and description thereof will not be repeated. In describing the example embodiments, when it is determined that a detailed description of well-known technology relating to the present disclosure unnecessarily makes the gist of the example embodiments obscure, the detailed description thereof will be omitted.

In addition, in describing constituent elements of the example embodiments according to the present disclosure, terms such as first, second, “A”, “B”, (a), (b), and the like may be used. These terms are merely intended to distinguish one constituent element from another constituent element, and the terms do not limit the nature, sequence or order of the corresponding constituent elements.

Constituent elements that have the same functions as constituent elements included in any of the example embodiments will be described using the same names in other example embodiments. Unless otherwise stated, description described in any of the embodiments may be applied to other embodiments, and the specific description will be omitted in the overlapping range.

According to an aspect of the present disclosure, there is provided a composition for dyeing a bentonite, the composition including a polyphenol and a mordant.

According to an embodiment of the present disclosure, the polyphenol may be at least one of lignin, pectin, tannin, and catechin.

According to an embodiment of the present disclosure, the mordant may be at least one of Fe³⁺, Fe²⁺ and Al³⁺.

According to an embodiment of the present disclosure, a molar ratio of the polyphenol to the mordant may range from 1:1 to 1:10.

According to another aspect of the present disclosure, there is provided a bentonite dyed by a composition for dyeing a bentonite, the composition including a polyphenol and a mordant.

According to an embodiment of the present disclosure, the dyed bentonite may have a crystalline spherical shape and a diameter of 20 to 50 μm.

According to another aspect of the present disclosure, there is provided a cosmetic composition containing a bentonite dyed by a composition for dyeing a bentonite, the composition including a polyphenol and a mordant.

According to an embodiment of the present disclosure, the dyed bentonite may have a crystalline spherical shape and a diameter of 20 to 50 μm.

According to another aspect of the present disclosure, there is provided a method for dyeing a bentonite, the method including steps of separating and purifying a bentonite; mixing a polyphenol and a mordant to prepare a solution; and stirring a mixed solution of the polyphenol and the mordant and a separated and purified bentonite.

According to an embodiment of the present disclosure, the separated and purified bentonite may have a particle size of 0.01 to 100 μm.

According to an embodiment of the present disclosure, the bentonite may be included in an amount of 1 to 20 wt % based on 100 wt % of the mixed solution of the polyphenol and the mordant.

According to an aspect of the present disclosure, there is provided a composition for dyeing a bentonite, the composition including a polyphenol and a mordant.

In the specification of the present disclosure, the term “polyphenol” refers to a polymer having a plurality of phenol functional groups. A polyphenol is a kind of chemicals found in plants and is characterized by two or more phenolic groups per molecule. Polyphenols are generally classified as Tannin and phenylpropanoids (flavonoids, Lignin, etc.). A phenol has a hydroxy group with which one hydrogen atom of a benzene is substituted, and a polyphenol has two or more hydroxy groups with which two or more hydrogen atoms of a benzene are substituted. There are thousands of types of polyphenols, including catechins in green tea, resveratrol in wine, and quercetin in apples and onions. Flavonoids abundant in fruits and isoflavones abundant in beans are also types of polyphenols.

According to an embodiment of the present disclosure, the polyphenol may be at least one of lignin, pectin, tannin, and catechin.

In the specification of the present disclosure, the term “mordant” means a metal salt used for mordanting. Mordanting is a process that helps coloring and adhesion of a dye solution by treating an object to be dyed before and after dyeing. Mordanting enhances binding property between an object to be dyed and a dye, enhances dyeing fastness, promotes dye adsorption, and increases chromaticity. Mordanting include pre-mordanting before dyeing; post mordanting after dyeing; simultaneous mordanting, which is dyed simultaneously with mordanting by adding a mordant to a dye solution; and non-mordanting that does not perform mordanting depending on dyes. Examples of the mordants to be mainly used include aluminum salts (alum, aluminum acetate, etc.), chromium salts (chromium alum, dichromate, chromium chloride, etc.), and iron salts (iron sulfate, iron acetate, etc.). In many cases, a color varies depending on the type of metal ion. Metal salts combine with dyes to become insoluble lakes that may adhere to a clay, a bentonite, fibers, and the like.

According to an embodiment of the present disclosure, the mordant may be at least one of Fe³⁺, Fe²⁺ and Al³⁺.

In the present disclosure, a polyphenol is a plant-derived material and is chelated with a mordant (Fe³⁺, Fe²⁺, Al³⁺, etc.) to show a color. The metal mordant such as Fe³⁺, Fe²⁺, and A1³+mediates adsorption and adhesion of polyphenol-metal mordant molecules on a bentonite, so that the color of the bentonite is changed by coloring.

According to an embodiment of the present disclosure, a molar ratio of the polyphenol to the mordant may range from 1:1 to 1:10. When the molar ratio of the polyphenol to the mordant may range from 1:1 to 1:10, the mordant adsorbs and adheres polyphenol-mordant molecules on a bentonite with high efficiency, and may maintain the coloring effect of a bentonite.

According to an embodiment of the present disclosure, a natural bentonite may be colored by a composition for dyeing a bentonite, the composition including a polyphenol and a mordant.

According to another aspect of the present disclosure, there is provided a bentonite dyed by a composition for dyeing a bentonite, the composition including a polyphenol and a mordant.

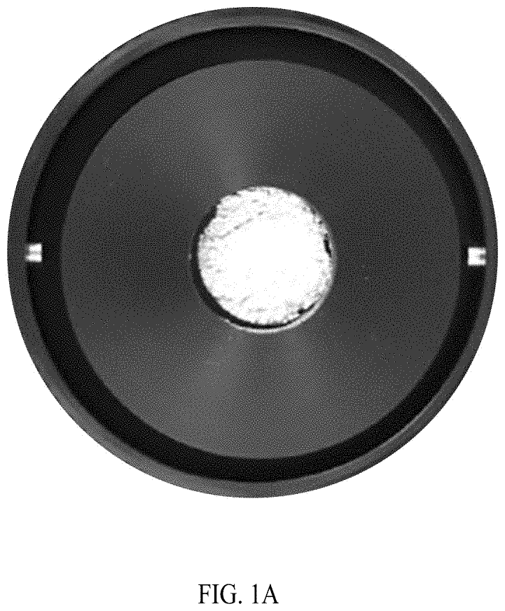

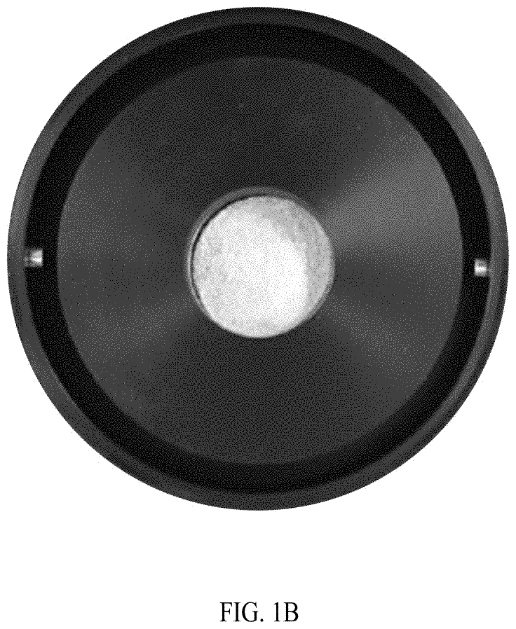

Referring to FIG. 1A, a purified natural bentonite shows white color. Referring to FIG. 1B, a bentonite dyed using a polyphenol extracted from pine needles and Fe³⁺ as a mordant shows colors close to white, red/purple and yellow.

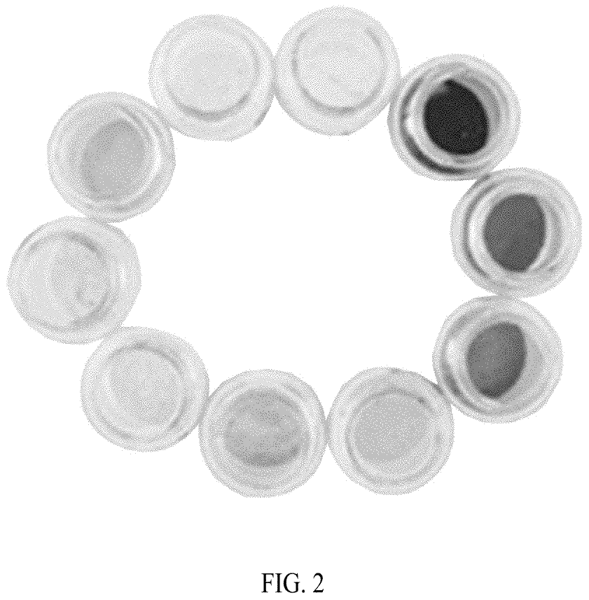

Referring to FIG. 2, various colors of a bentonite dyed with a solution prepared by mixing 10 mM polyphenol and 100 mM mordant are shown.

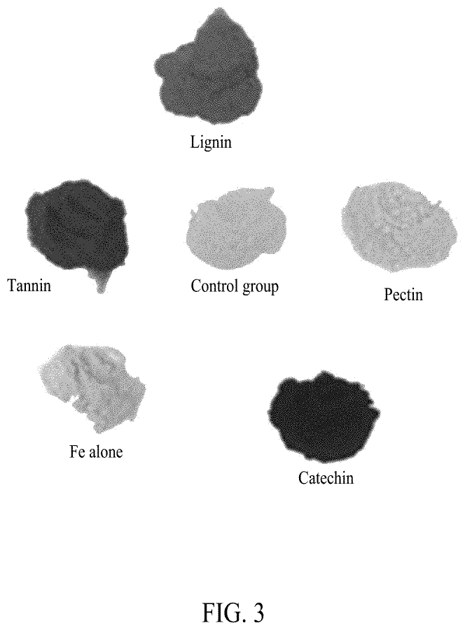

Referring to FIG. 3, various colors of a bentonite dyed with a solution prepared by mixing a polyphenol having a weight of 10 mg and a mordant having a molarity of 50 mM are shown.

According to an embodiment of the present disclosure, the dyed bentonite may have a crystalline spherical shape and a uniform diameter upon spray drying. The uniform diameter may be 10 to 100 μm, and preferably 20 to 50 μm. The dyed bentonite having a crystalline spherical shape and a uniform diameter has good skin texture, so it may be applied to cosmetic compositions and the like.

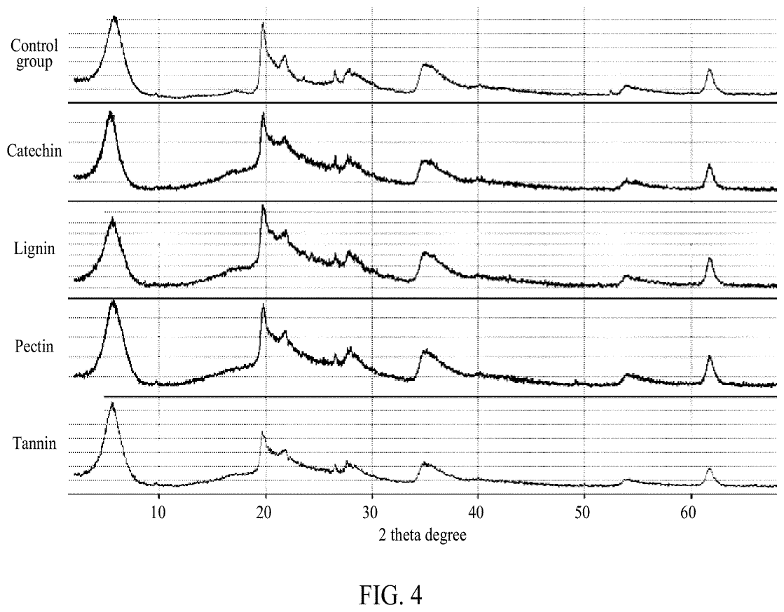

According to FIG. 4, as a result of XRD analysis, even if polyphenol-metal mordant molecules are adsorbed or adhered on a bentonite, mineralogical properties of a bentonite are maintained. Accordingly, the metal mordant changes the color of a bentonite by mediating adsorption and adhesion of polyphenol-metal mordant molecules on a bentonite, but the polyphenol-metal mordant molecules do not destroy mineralogical properties of a bentonite.

The cosmetic composition may be prepared in any one or more formulations of an atopy soap, a cleansing foam, a cleansing cream, a cleansing water, a bath agent, a skin lotion, a skin softener, a skin toner, a lotion, a cream, an essence, an astringent, an emulsion, a gel, a lipstick, a spray, a shampoo, a conditioner, a treatment, a body cleanser, a pack, a massage agent, a face powder, a compact, a foundation, a two-way cake, and a makeup base. As long as it is an article to which a clay mineral such as a dyed bentonite may be applied, it is not limited to the above.

According to another aspect of the present disclosure, there is provided a method for dyeing a bentonite, the method including steps of separating and purifying a bentonite; mixing a polyphenol and a mordant to prepare a solution; and stirring a mixed solution of the polyphenol and the mordant and a separated and purified bentonite.

According to an embodiment of the present disclosure, the separated and purified bentonite may have a particle size of 0.01 to 100 μm.

According to an embodiment of the present disclosure, the bentonite may be included in an amount of 1 to 20 wt % based on 100 wt % of the mixed solution of the polyphenol and the mordant.

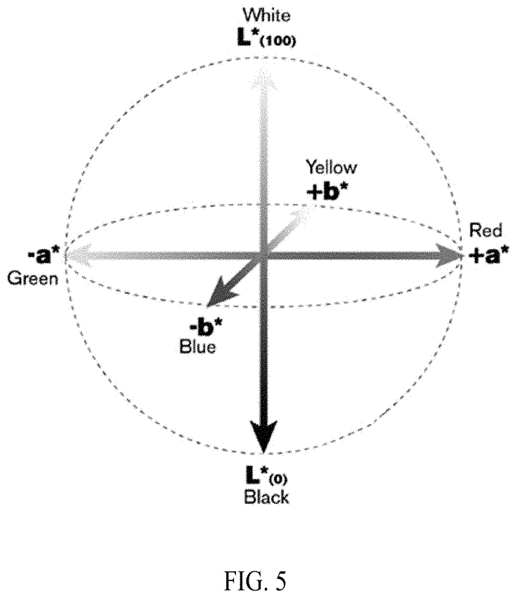

A bentonite was dyed using the composition for dyeing a bentonite of the present disclosure, and analyzed using CIE L*a*b* color space (FIG.5).

In the CIE L*a*b* color space, a value L* represents lightness. L*=0 indicates black, and L*=100 indicates white. A value a* indicates whether the value is toward red or green. When the value is negative number, the value is toward green and when the value is positive number, the value is toward red/purple. A value b* indicates blue or yellow When the value is negative number, the color is blue and when the value is positive number, the color is yellow.

### EXPERIMENTAL EXAMPLE 1.

### Purification of Bentonite

A bentonite ore was pulverized and washed with water. After washing, it was sieved with a wet classifier to separate a bentonite having a particle size of 25 μm or less. At this time, a solid-liquid ratio of a suspension entering the classifier is within 10%. A suspension having a solid -liquid ratio of 5% or less was prepared by mixing a separated bentonite having a particle size of 25 μm or less with deionized or purified water. pH of the bentonite suspension was adjusted so that pH of the bentonite suspension having a basicity was 6 to 8 using an organic acid or an inorganic acid. A coagulant was added thereto, followed by stirring. As the coagulant, a CaCl₂ solution is preferable, and a concentration thereof is 1M or less. After stirring, solid-liquid separation was performed, and the resultant was washed to a chlorine concentration of 200-300 ppm or less, so that the concentration of the final suspension was less than 5%.

### <EXPERIMENTAL EXAMPLE 2

### Preparation of Composition for Dyeing Bentonite and Bentonite Dyeing

Preparation of Dyeing Solution according to Molarity of Polyphenol and Bentonite Dyeing

A solution for dyeing a bentonite was prepared by mixing a polyphenol and a mordant. The polyphenol is at least one of lignin, pectin, tannin, or catechin, and the mordant is at least one of Fe³⁺, Fe²⁺, or Al³⁺. A molar ratio of the polyphenol to the mordant is 1:1 to 1:10. A polyphenol having a molarity of 10 mM and a mordant having a molarity of 100 mM in Table 1 were mixed. In Preparation Examples 1 to 6, solutions for dyeing a bentonite were prepared using a polyphenol having a molarity of 10 mM.

TABLE 1

| | | Polyphenol | | Mordant | |
|---|---|---|---|---|---|
| Division | Volume | Types | Molarity | Types | Molarity |
| Preparation | 25 ml | Lignin | 10 mM | Fe³⁺ | 100 mM |
| example 1 | | | | | |
| Preparation | 25 ml | Pectin | 10 mM | | 100 mM |
| example 2 | | | | | |
| Preparation | 25 ml | Tannin | 10 mM | | 100 mM |
| example 3 | | | | | |
| Preparation | 25 ml | Catechin | 10 mM | | 100 mM |
| example 4 | | | | | |
| Preparation | 25 ml | Pine needle | 10 mM | Fe³⁺ | 100 mM |
| example 5 | | extract* | | | |
| Preparation | 25 ml | Pine needle | 10 mM | Fe²⁺ | 100 mM |
| example 6 | | extract* | | | |

Pine needle extract is a solution extracted by alcohol. 5g of pine needles and 30 ml of alcohol (99.8% or more) are used, and stirring is performed for more than 6 hours.

TABLE 2

| Division | Bentonite | Polyphenol | Mordant |
|---|---|---|---|
| Weight or Molarity | 0.5 g | 10 mM | 100 mM |

25 ml of the dyeing solution prepared according to Preparation Examples 1 to 6 of Table 1 and 0.5 g of the purified bentonite (Table 2) were stirred. At this time, a solid-liquid ratio (bentonite/solution (mordant+polyphenol)) of a bentonite is 5 to 10%. Stirring may be performed for 1 to 6 hours, and stirring time may increase as a volume of a solution obtained by mixing a mordant and a polyphenol increases.

Preparation of Dyeing Solution according to Weight of Polyphenol and Bentonite Dyeing

A solution for dyeing a bentonite was prepared by mixing a polyphenol having a weight of 10 mg and a mordant having a molarity of 50 mM in Table 3. The polyphenol is at least one of lignin, pectin, tannin, or catechin, and the mordant is at least one of Fe³⁺, Fe²⁺, or Al³⁺. In Preparation Examples 7 to 11, solutions for dyeing a bentonite were prepared by a polyphenol having a weight of 10 mg.

TABLE 3

| | | Polyphenol | | Mordant | |
|---|---|---|---|---|---|
| Division | Volume | Types | Weight | Types | Molarity |
| Preparation | 20 ml | — | — | Fe³⁺ | 50 mM |
| example 7 | | | | | |
| Preparation | 20 ml | Lignin | 10 mg | | 50 mM |
| example 8 | | | | | |
| Preparation | 20 ml | Pectin | 10 mg | | 50 mM |
| example 9 | | | | | |
| Preparation | 20 ml | Tannin | 10 mg | | 50 mM |
| example 10 | | | | | |
| Preparation | 20 ml | Catechin | 10 mg | | 50 mM |
| example 11 | | | | | |

TABLE 4

| Division | Bentonite | Polyphenol | Mordant |
|---|---|---|---|
| Weight or Molarity | 0.5 g | 10 mg | 50 mM |

The dyeing solution prepared according to Preparation Examples 7 to 11 and a bentonite purified before dyeing were acid-washed with a 0.5 M HC1 solution for 30 min. Then, 20 ml of the dyeing solution prepared according to Preparation Examples 7 to 11 and 0.5 g of the purified bentonite (Table 4) were stirred.

### EXAMPLE 1

### Color Analysis of Dyed Bentonite

The purified bentonite (0.5 g) in Experimental Example 1 was dyed with the dyeing solution containing a polyphenol and a mordant prepared in Experimental Example 2, and the color change was analyzed using a colorimeter (produced by MINOLTA). Color of dyed bentonite (0.5g) was analyzed using the CIE L*a*b* color space. Table 5 shows the result of analyzing change in color of a bentonite using a colorimeter, in which the bentonite is dyed with a solution prepared by fixing a molarity of polyphenol to 10 mM. Table 6 shows the result of analyzing change in color of a bentonite using a colorimeter, in which the bentonite is dyed with a solution prepared by fixing a weight of polyphenol to 10 mg.

TABLE 5

| | Division | L* | a* | b* |
|---|---|---|---|---|
| Control group | Purified | 74.69 | −0.37 | 6.14 |
| | bentonite | | | |
| Preparation | Lignin, Fe³⁺ | 14.34 | 1.22 | 6.1 |
| example 1 | | | | |
| Preparation | Pectin, Fe³⁺ | 68.38 | 4.4 | 24.01 |
| example 2 | | | | |
| Preparation | Tannin, Fe³⁺ | 4.68 | 0.4 | −0.44 |
| example 3 | | | | |
| Preparation | Catechin, Fe³⁺ | 15.32 | 1.47 | 5.53 |
| example 4 | | | | |
| Preparation | Pine needle | 71.3 | 4.96 | 35.68 |
| example 5 | extract, Fe³⁺ | | | |
| Preparation | Pine needle | 73.82 | 0.87 | 16.83 |
| example 6 | extract, Fe²⁺ | | | |

A control group was a purified bentonite, which was analyzed using a colorimeter. The chromaticity has a value L of 74.69, a value a of -0.37, and a value b of 6.14 and shows colors close to white, green, and yellow.

In Preparation Example 1, a dyeing solution was prepared by mixing 10 mM lignin and 100 mM Fe³⁺, with which the purified bentonite was dyed. As a result of analysis using a colorimeter, the chromaticity has a value L of 14.34, a value a of 1.22 and a value b of 6.1. The value b was almost unchanged, but the values L and a were changed compared to the chromaticity of the purified bentonite and shows colors close to black and red/purple.

In Preparation Example 2, a dyeing solution was prepared by mixing 10 mM pectin and 100 mM Fe³⁺, with which the purified bentonite was dyed. As a result of analysis using a colorimeter, the chromaticity has a value L of 68.38, a value a of 4.4, and a value b of 24.01 and shows colors close to white, red/purple and yellow.

In Preparation Example 3, a dyeing solution was prepared by mixing 10 mM tannin and 100 mM Fe³⁺, with which the purified bentonite was dyed. As a result of analysis using a colorimeter, the chromaticity has a value L of 4.68, a value a of 0.4, and a value b of -0.44, in which the value L is significantly changed compared to chromaticity of the purified bentonite. The chromaticity shows colors close to black, red/purple, and blue.

In Preparation Example 4, a dyeing solution was prepared by mixing 10 mM catechin and 100 mM Fe³⁺, with which the purified bentonite was dyed. As a result of analysis using a colorimeter, the chromaticity has a value L of 15.32, a value of 1.47, and a value b of 5.53, and shows colors close to black, red/purple and yellow.

In Preparation Example 5, a dyeing solution was prepared by mixing 10 mM pine needle extract and 100 mM Fe³⁺, with which the purified bentonite was dyed. As a result of analysis using a colorimeter, the chromaticity has a value L of 71.3, a value of 4.96, and a value b of 35.68, and shows colors close to white, red/purple and yellow.

In Preparation Example 6, a dyeing solution was prepared by mixing 10 mM pine needle extract and 100 mM Fe²⁺, with which the purified bentonite was dyed. As a result of analysis using a colorimeter, the chromaticity has a value L of 73.82, a value of 0.87, and a value b of 16.83 and shows colors close to white, red/purple and yellow.

TABLE 6

| | Division | L* | a* | b* |
|---|---|---|---|---|
| Control group | Acid-washed | 64.51 | −2.03 | 6.01 |
| | bentonite | | | |
| Preparation | Fe³⁺ | 61.61 | −5.85 | 37.33 |
| example 7 | | | | |
| Preparation | Lignin, Fe³⁺ | 39.01 | −0.18 | 14.03 |
| example 8 | | | | |
| Preparation | Pectin, Fe³⁺ | 66.27 | −7.30 | 30.27 |
| example 9 | | | | |
| Preparation | Tannin, Fe³⁺ | 34.04 | −3.80 | 7.85 |
| example 10 | | | | |
| Preparation | Catechin, Fe³⁺ | 29.41 | 1.36 | 8.99 |
| example 11 | | | | |

As a control group, a purified bentonite was acid-washed with a 0.5 M HCl solution for 30 min, and analyzed using a colorimeter. The chromaticity has a value L of 64.51, a value a of −2.03, and a value b of 6.01 and shows colors close to white, green, and yellow.

In Preparation Example 7, a dyeing solution contains 50 mM Fe³⁺, with which the acid-washed purified bentonite was dyed. As a result of analysis using a colorimeter, the chromaticity has a value L of 61.61, a value a of -5.85, and a value b of 37.33. The values L and a were almost unchanged, but the value b changed compared to the chromaticity of the purified bentonite, resulting in yellow color darker than that of the acid-washed purified bentonite.

In Preparation Example 8, a dyeing solution was prepared by mixing 10 mg of lignin and 50 mM Fe³⁺, with which the purified bentonite was dyed. As a result of analysis using a colorimeter, the chromaticity has a value L of 39.01, a value of -0.18, and a value b of 14.03 and shows colors close to black, green, and yellow.

In Preparation Example 9, a dyeing solution was prepared by mixing 10 mg of pectin and 50 mM Fe³⁺, with which the acid-washed purified bentonite was dyed. As a result of analysis using a colorimeter, the chromaticity has a value L of 66.27, a value of -7.30, and a value b of 30.27 and shows colors close to white, green, and yellow.

In Preparation Example 10, a dyeing solution was prepared by mixing 10 mg of tannin and 50 mM Fe³⁺, with which the acid-washed purified bentonite was dyed. As a result of analysis using a colorimeter, the chromaticity has a value L of 34.04, a value of -3.80, and a value b of 7.85 and shows colors close to black, green, and yellow.

In Preparation Example 11, a dyeing solution was prepared by mixing 10 mg of catechin and 50 mM Fe³⁺, with which the acid-washed purified bentonite was dyed. As a result of analysis using a colorimeter, the chromaticity has a value L of 29.41, a value of 1.36, and a value b of 8.99 and shows colors close to black, red/purple, and yellow.

Although the example embodiments have been described by limited drawings as described above, a person having ordinary skill in the art may apply various modifications and changes based on the example embodiments. For example, although described techniques are performed in a different order from a described method, and/or described elements such as systems, structures, devices, and circuits are combined or merged in a different form from the described method, or replaced or substituted with other elements or equivalents, appropriate results may be achieved.

Therefore, equivalents to other example embodiments, other example embodiments, and patent claims also belong to the scope of claims to be described below.

What is claimed is:

1. A composition for dying a bentonite, the composition comprising a polyphenol and a mordant.

2. The composition of claim 1, wherein the polyphenol is at least one selected from a group consisting of lignin, pectin, tannin, and catechin.

3. The composition of claim 1, wherein the mordant is at least one selected from a group consisting of Fe³⁺, Fe²⁺ and Al³⁺.

4. The composition of claim 1, wherein a molar ratio of the polyphenol to the mordant ranges from 1:1 to 1:10.

5. A bentonite dyed by a composition for dyeing a bentonite, the composition comprising a polyphenol and a mordant.

6. The bentonite of claim 5, wherein the dyed bentonite has a crystalline spherical shape and a diameter of 20 to 50 μm.

7. A method for dyeing a bentonite, the method comprising:
separating and purifying a bentonite;
mixing a polyphenol and a mordant to prepare a solution; and
stirring a mixed solution of the polyphenol and the mordant and a separated and purified bentonite.

8. The method of claim 7, wherein the separated and purified bentonite has a particle size of 0.01 to 100 μm.

9. The method of claim 7, wherein the bentonite is included in an amount of 1 to 20% by weight (wt %) based on 100 wt % of the mixed solution of the polyphenol and the mordant.
